(19) United States
(12) Patent Application Publication
(10) Pub. No.: US 2019/0365697 A1
(43) Pub. Date: Dec. 5, 2019

(54) Method for Treating Liver Dysfunction

(71) Applicant: PROFEAT BIOTECHNOLOGY CO., LTD., Taoyuan (TW)

(72) Inventors: Kai-Ting WANG, Taoyuan (TW); Tsun-Yuan LIN, Taoyuan (TW); Mu-Kuei CHEN, Taoyuan (TW); Hsun-Jin JAN, Taoyuan (TW)

(21) Appl. No.: 16/485,604

(22) PCT Filed: Feb. 17, 2017
(86) PCT No.: PCT/CN2017/073865

§ 371 Date: Aug. 13, 2019

(51) Int. Cl. A61K 31/295; A61K 31/198; A61P 1/16

(52) U.S. Cl. CPC A61K 31/295; A61K 9/08; A61P 1/16; A61K 31/198

(57) ABSTRACT

The present invention provides use of a composition comprising ferrous amino acid chelate for the manufacture of a medicament for treatment of liver dysfunction, wherein the medicament comprises an effective amount of the ferrous amino acid chelate composition and a pharmaceutically acceptable carrier. The present invention proves that the serum biochemistry values related to the liver function can be reduced by administering the composition comprising ferrous amino acid chelate. For animals whose liver functions cannot be improved by long-term administration of S-adenosylmethionine, their liver functions can be effectively improved by administration of the composition comprising ferrous amino acid chelate of the present invention instead. Furthermore, the composition comprising ferrous amino acid chelate can improve the functions of an aged liver.

### BACKGROUND OF THE INVENTION

### 1. Field of the Invention

The present invention relates to use of a composition comprising ferrous amino acid chelate, and particularly the use for treatment of liver dysfunction.

### 2. Description of the Prior Arts

Liver function test is one of the most common clinical tests. Besides, blood test is one of the most important methods to determine whether liver diseases occur or not. The items for liver function test are numerous, and the main purposes of liver function tests are as follows: (1) to detect the liver diseases, (2) to diagnose the liver diseases and to investigate the probable diseases, (3) to evaluate the severity of the liver diseases, and (4) to treat and to predict and track the prognosis.

Glutamic oxaloacetic transaminase (GOT), also known as aspartate aminotransferase (AST), and glutamic-pyruvic transaminase (GPT), also known as alanine transaminase (ALT), are very important for detecting the damage to hepatocytes. Once hepatocytes are damaged or necrosis occurs, AST and ALT are released into blood and the levels thereof are increased. ALT are mainly derived from the liver, while AST is normally found in tissues other than liver, for example, heart, skeletal muscle, kidney, etc. Normally, the levels of serum AST and ALT are increased or decreased in a consistent manner. Which level would be higher depends on the cause of disease and the disease process.

Serum contains alkaline phosphatase (ALKP) from various organs, including liver, bones, small intestine, placenta, etc. When the level of alkaline phosphatase is increased, it should be firstly clarified whether the case involves pregnancy, developing adolescents or the rare lymphoproliferative disease in the small intestinal. Next, it should be identified whether the origin is the liver and gall system or the skeleton system. Elevated alkaline phosphatase results from the increased synthetase in hepatocytes and cholangiocytes. Normally, mild and medium increase (1-fold to 2-fold higher than the normal level) can be found in the patients of hepatitis, metastasis of cirrhosis or infiltrating liver disease, such as leukemia. Significant increase (3-fold to 10-fold higher than the normal level) can be found in the extrahepatic bile duct obstruction or intrahepatic cholestasis, such as drug-induced or primary biliary cirrhosis.

Cholesterol (CHOL) is widely distributed in the animal bodies, and is especially rich in the brain and neural tissue. The levels of cholesterol in the kidney, spleen, skin, liver and bile are also high. It is an essential substance for tissues and cells being involved in cell membrane formation and the raw material for the synthesis of bile acid, vitamin D, and steroid hormones. The increase of serum cholesterol can be found in diseases such as diabetes, simple obstructive jaundice, obesity, high fat diet, hypothyroidism, Cushing's syndrome, nephrotic syndrome, etc. The decrease of serum cholesterol can be found in conditions such as severe malnutrition, malignant tumors, severe damage of hepatocytes, and protein-losing enteropathy. Liver and kidney diseases or the endocrine dysfunction result in the increase of cholesterol. And the hypothyroidism of dogs leads to 70% probability of elevated cholesterol level.

Total bilirubin (TBIL) includes the direct bilirubin and indirect bilirubin. The normal serum bilirubin is basically derived from hemoglobin resulting from the breakdown of senescent red blood cells. When liver diseases occur, bilirubin cannot be converted into bile or the intrahepatic cholestasis, due to turgidity of hepatocytes, causes the increase of both the direct bilirubin and the indirect bilirubin and leads to hepatocellular jaundice. When cholecystitis or the obstruction of common biliary tract occurs, bile flow into duodenum is obstructed, which causes the obstructive jaundice. Both the direct bilirubin and indirect bilirubin are increased when hepatocellular jaundice occurs. When occurrence of hepatocellular jaundice is suspected, the diagnosis is made after combining indexes of ALT, AST, and ALKP and various clinical symptoms. When hepatocellular jaundice occurs, the total bilirubin is proportional to the severity of the liver disease in a certain degree. When the total bilirubin level indicates the sever jaundice, it is called “biliary enzyme separation” if the levels of ALT and AST are not increased that much. The reason of “biliary enzyme separation” is the failure in converting indirect bilirubin into bile in the case of severe damages of hepatocytes, which results in intrahepatic cholestasis. There are only few active hepatocytes and thus the released enzyme is reduced accordingly. Therefore, the levels of ALT and AST can be used as an alert of an extremely severe liver damage, the middle or late stage of cirrhosis and liver failure. Obstructive jaundice means that the hepatocytes combine indirect bilirubin to form the direct bilirubin and produce the bile. However, the release of bile is obstructed, which causes the symptoms of an increased level of direct bilirubin, and a normal level of indirect bilirubin or a mildly increased level of indirect bilirubin. Obstructive jaundice is mainly caused by the bile duct obstruction and compression due to gallstones, parasites, liver tumors, cholecystic tumors, or pancreatic gland tumors. In this situation, the level of ALT is mildly elevated, the level of AST is normal or mildly elevated, and the level of ALKP is significantly elevated in general.

When the liver is damaged, the function of liver is highly impacted by the reduced glutathione due to the reduced production of S-adenosylmethionine (SAMe). Research reports indicate that the content of glutathione is reduced in most of the liver problems. Usually, it is difficult to find the real reason when the liver does not function properly. Therefore, in any case, the regular care to improve liver functions is very important. In the prior art, in order to improve liver functions, SAMe is administered to dogs and cats to increase the content of glutathione. However, the recent practices showed that administration of SAMe to animals suffering from liver dysfunction cannot improve the liver functions.

In view of this, it is necessary to improve the prior arts to develop a composition for the improvement of the liver function.

### SUMMARY OF THE INVENTION

In view of the shortcoming about side effects of the conventional medicines, the objective of the present invention is to provide use of a composition comprising ferrous amino acid chelate for the manufacture of a medicament for the treatment of liver dysfunction, wherein the composition comprising ferrous amino acid chelate has the effect of regulating liver functions.

To achieve the above purpose, the present invention adopts the technical means of providing use of a composition comprising ferrous amino acid chelate for the manufacture of a medicament for treatment of liver dysfunction. Said medicament comprises an effective amount of the composition comprising ferrous amino acid chelate and a pharmaceutically acceptable carrier.

According to the present invention, the term “composition comprising ferrous amino acid chelate” refers to a composition comprising ferrous amino acid chelate prepared by mixing inorganic iron and amino acid.

Preferably, the chelating ratio of ferrous to amino acid of the ferrous amino acid chelate in the composition comprising ferrous amino acid chelate is between 1:1 and 1:4.

Preferably, the chelating ratio of ferrous to amino acid of the ferrous amino acid chelate in the composition comprising ferrous amino acid chelate is between 1:1.5 and 1:2.5.

Preferably, the effective amount of the composition comprising ferrous amino acid chelate for humans is between 0.5 mg/kg/day and 4 mg/kg/day. The aforementioned dosage is calculated in accordance with the guidance document “Estimating the maximum safe starting dose in initial clinical trials for therapeutics in adult healthy volunteers” published by the U.S. Food and Drug administration in 2005.

Preferably, the effective amount of the composition comprising ferrous amino acid chelate for mice is between 5 mg/kg/day and 20 mg/kg/day.

Preferably, the effective amount of the composition comprising ferrous amino acid chelate for dogs is between 2 mg/kg/day and 5 mg/kg/day.

More preferably, the composition comprising ferrous amino acid chelate is prepared by mixing inorganic iron and amino acid and heating at 60° C. to 90° C. for 8 hours to 48 hours, wherein the weight ratio of inorganic iron to amino acid is between 1:1.2 and 1:1.5.

More preferably, the inorganic iron is ferrous sulfate, ferrous chloride, ferrous pyrophosphate or any combination thereof; and the amino acid is glycine.

More preferably, the composition comprising ferrous amino acid chelate contains 95 wt % to 100 wt % ferrous glycinate chelate. More preferably, the composition comprising ferrous amino acid chelate contains 98 wt % to 99.9 wt % ferrous glycinate chelate.

More preferably, the composition comprising ferrous amino acid chelate further includes a reductant. The reductant includes, but is not limited to, ascorbic acid, citric acid, acetic acid, propionic acid, butyric acid, lactic acid, malic acid, sulfonic acid, succinic acid, and any combination thereof.

According to the present invention, the term “effective amount” refers to a dosage which is effective to reduce the serum biochemistry values related to the liver function in a given period of time. According to the present invention, the effective amount refers to an amount in a specific range in which administration of the composition comprising ferrous amino acid chelate reduces the serum biochemistry values related to the liver function, ALT, AST and ALKP, or reduces the values to the normal ranges. According to the present invention, it also refers to the improvement of the functions of an aged liver.

According to the present invention, the term “liver function” refers to the determination whether the liver function is normal or not by examining the serum biochemistry values such as the values of serum ALT and AST. Additionally, ALKP can also be used as an auxiliary basis for determination. Among others, the reference standard value for ALT ranges from 10 U/L to 100 U/L, and the reference standard value for AST ranges from 0 U/L to 50 U/L.

According to the present invention, the “pharmaceutically acceptable carriers” include, but are not limited to, solvents, emulsifiers, suspending agents, decomposers, binding agents, excipients, stabilizing agents, diluents, gelling agents, preservatives, lubricants, surfactants and other similar carriers or the carriers that are suitable for the present invention.

In accordance with the present invention, the “medicament” can be present in various forms, including, but not limited to, liquid, semi-solid and solid dosage forms, such as liquid solutions, emulsions, suspensions, powders, tablets, pills, lozenges, troches, chewing gums, capsules, liposomes, suppositories, and the other similar dosage forms or the dosage forms that are suitable for the present invention.

Preferably, the medicament is in an enteral or parenteral dosage form.

More preferably, said enteral dosage form is an oral dosage form, which is solutions, emulsions, suspensions, powders, tablets, pills, lozenges, troches, chewing gums, or capsules.

The advantage of the present invention is that the composition comprising ferrous amino acid chelate of the present invention has the effect of reducing the serum biochemistry values related to the liver function, ALT, AST and ALKP. For animals whose liver function cannot be improved by long-term administration of SAMe, the liver function thereof can be effectively improved by administration of the composition comprising ferrous amino acid chelate of the present invention instead. Furthermore, the composition comprising ferrous amino acid chelate of the present invention can improve the functions of an aged liver.

### BRIEF DESCRIPTION OF THE DRAWINGS

The following figures are only for illustrative and explanatory purposes, and are not intended to limit the scope of the present invention.

FIG. 1 is a line graph of dogs' ALT in a severe group and a mild group after continuous administration of Composition A1 of the present invention.

FIG. 2 is a line graph of dogs' AST in the mild group after continuous administration of Composition A1 of the present invention.

FIG. 3 is a bar chart of dogs' ALT after discontinued administration of Composition A1 of the present invention.

FIG. 4 is a bar chart of dogs' AST after discontinued administration of Composition A1 of the present invention.

FIG. 5 is a bar chart of aged mice's AST after administration of Composition A1 of the present invention.

### DETAILED DESCRIPTION OF THE PREFERRED EMBODIMENTS

The technical features adopted in the present invention in order to achieve the purpose are further explained through the preferred embodiments below in combination with the figures.

### Preparation Example 1: Preparation of the Composition Comprising Ferrous Amino Acid Chelate

The composition comprising ferrous amino acid chelate was prepared as follows. Ferrous sulfate and glycine (more than 98% purity) were mixed in a weight ratio of 1:1.3 and heated at 60° C. to 90° C. for 8 hours to 48 hours to obtain the composition comprising ferrous amino acid chelate, wherein the chelating ratio of ferrous to amino acid of the ferrous amino acid chelate was between 1:1 and 1:4. Said composition was referred to as A1.

### Example 1 Continuous Administration of Composition A1 to Dogs

Eleven dogs suffering from the liver dysfunction were examined, and were administered with Composition A1 at the dosage of 27 mg/10 kg (2.7 mg/kg) per day. The serum biochemistry values related to the liver function, ALT, AST and ALKP, were detected after blood collection and then tracked without a fixed schedule for up to 160 days. Among others, the reference standard value for ALT ranges from 10 U/L to 100 U/L, and the reference standard value for AST ranges from 0 U/L to 50 U/L. The value on the day 0 serves as the data before administration of Composition A1. Three of the dogs, whose ALT values exceeded 500 U/L before administration of Composition A1, were classified as the severe group. The other 8 dogs, whose ALT values ranged from 100 U/L to 200 U/L before administration of composition A1, were classified as the mild group.

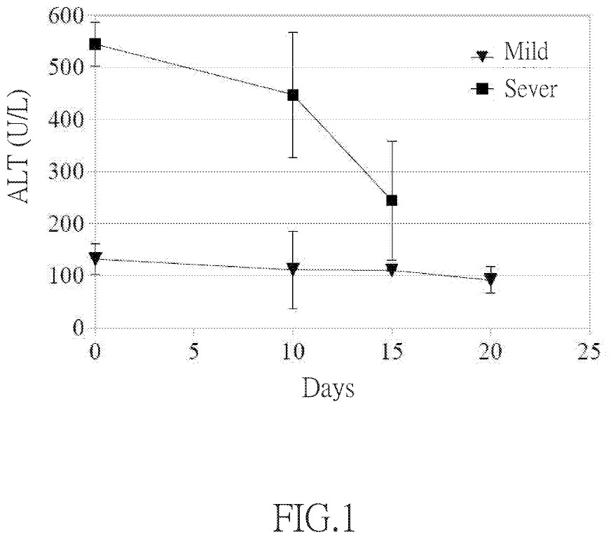

As shown is FIG. 1, 15 days after continuous administration of Composition A1 to the severe group, the average ALT value significantly decreased by up to 300 U/L, from 545 U/L to 245 U/L. The value of one of the dogs in the severe group even decreased by 200 U/L after 1 day administration. 20 days after continuous administration of Composition A1 to the mild group, the average ALT values was decreased to 92 U/L within the normal range. In addition, the value was maintained within the normal range after continuous administration for 3 to more than 5 months. This indicates that the administration of Composition A1 can effectively improve the abnormal ALT in dogs.

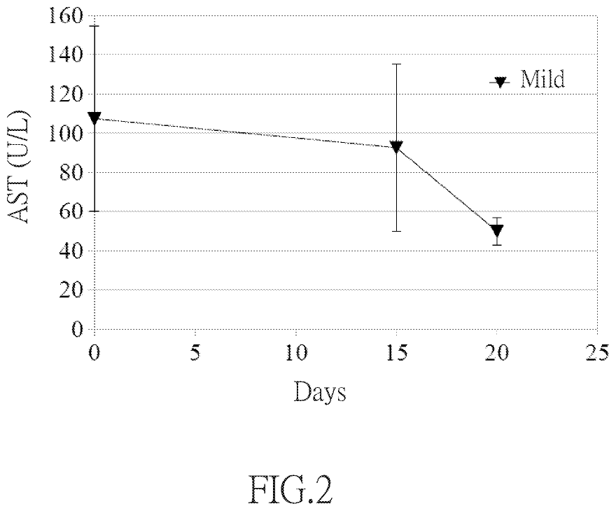

As shown in FIG. 2, the value of the mild group on the day 0 serves as the data before administration of Composition A1, and the AST values thereof were between 70 U/L and 200 U/L. After continuous administration of Composition A1 to mild group, the average value was decreased to 50 U/L within the normal range. And the value maintained within the normal range after continuous administration for more than 3 months. This indicates that the administration of Composition A1 can effectively improve the abnormal AST in dogs.

After continuous administration of Composition A1 for 15 days, the ALKP value of one of the dogs suffering from the liver dysfunction in the severe group decreased from more than 2000 U/L to 705 U/L. The decreasing extent is more than 1300 U/L. The ALKP value of another dog after continuous administration of Composition A1 for 18 days, decreased from more than 1834 U/L to 1035 U/L. The decreasing extent is up to about 800 U/L. This indicates that the administration of Composition A1 can effectively improve the abnormal ALKP in dogs.

### Example 2 Interruption of the Administration of Composition A1, Followed by the Resumed Administration

Five dogs suffering from the liver dysfunction were examined, and were administered the Composition A1 at the dosage of 27 mg/10 kg (2.7 mg/kg) per day. The administration of Composition A1 was stopped once the serum biochemistry values related to the liver function, ALT, AST and ALKP, detected by blood collection decreased greatly. After the serum biochemistry values related to the liver function, ALT, AST and ALKP, increased greatly, the administration the Composition A1 was resumed.

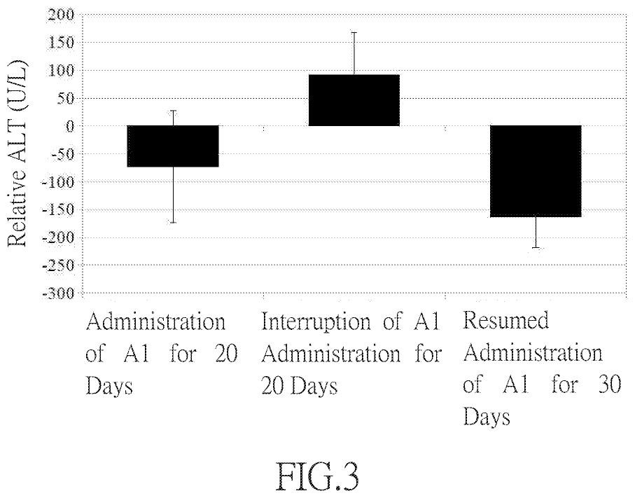

As shown in FIG. 3, dogs suffering from the liver dysfunction were administered with the Composition A1 for about 20 days in average, and the ALT value decreased by about 73 U/L was observed. However, the ALT value increased by about 92 U/L was observed from the blood collection 20 days after the administration of Composition A1 was interrupted. Therefore, the Composition A1 was administered again. The ALT value decreased by about 163 U/L was observed after 30 days of resumed administration of Composition A1.

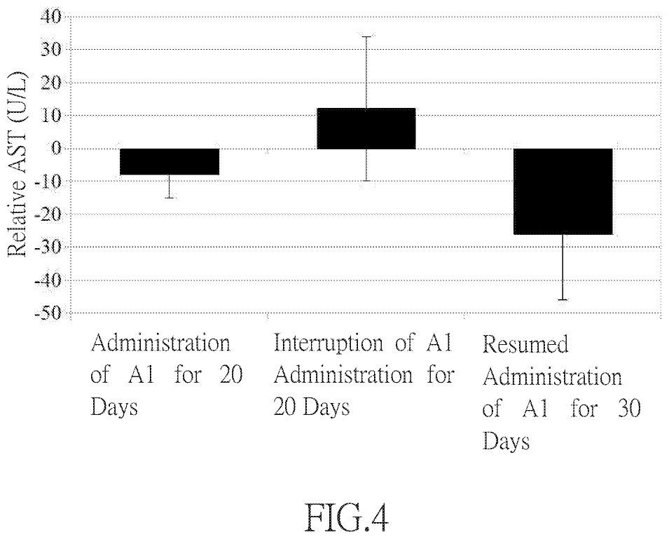

As shown in FIG. 4, dogs suffering from the liver dysfunction were administered with the Composition A1 for only about 20 days in average, and then the AST value decreased by about 8 U/L was observed. However, the AST value increased by about 12 U/L was observed from the blood collection 20 days after administration of Composition A1 was interrupted. Therefore, the Composition A1 was administered again. The AST value decreased by about 26 U/L is observed after 30 days of resumed administration of Composition A1.

### Example 3 Administration of SAMe Followed by Administration of Composition A1 to Dogs

It is clinically observed that after administration of the conventional medicine SAMe alone to dogs, the liver functions still cannot be improved. Therefore, Composition A1 was administered to 3 dogs, whose liver functions were not improved after the administration of SAMe. And the serum biochemistry values related to the liver function, ALT, AST and ALKP, were examined.

It can be observed that in dog No. 1, the ALT value decreased by about 45 U/L, and AST value decreased by about 50 U/L after administration of Composition A1 instead of SAMe for 4 months. Both the values fall within the standard range. The ALT value decreased by about 50 U/L in dog No. 2 was observed after administration of Composition A1 instead of SAMe for 20 days. The ALT value decreased by about 35 U/L of dog No. 3 was observed after administration of Composition A1 instead of SAMe for only 2 days. Therefore, for the doges whose liver function cannot be improved by long-term administration of SAMe, administration of Composition A1 instead effectively decreases the values related to the liver function in dogs.

### Example 4 Administration of Composition A1 to Aged Mice

Twelve-month-old mice (C57B/6J), used as the laboratory animal of aging model, were continuously administered at the dosage of 200 μg/mouse/day for (1) 4 months and (2) 6 months. The serum biochemistry values related to the liver function, ALT and AST, of the group administered with Composition A1 were determined and then were compared to control group.

(1) Administration for 4 Months

TABLE 1
The values of ALT and AST in respect of liver function

biochemistry values

| groups | AST | ALT |
|---|---|---|
| Control Group | 302.00 ± 52.16 | 41.00 ± 9.76 |
| A1 | 162.33 ± 53.00* | 43.00 ± 15.12 |

*represents P < 0.05 compared to control group

When the liver acute inflammatory occurs, the ALT value will be increased greatly. And when the liver chronic inflammatory occurs, the AST value will be increased constantly. According to Table 1, it can be observed that the liver of aged mice may already be aged and chronically inflamed. Thus, without acute inflammatory, the ALT value remained in the normal range while the AST value increased to 302 U/L. After administration of Composition A1 for 4 months, it can be observed that the ALT value remained in the normal range without changes, but the AST value dropped significantly. The value decreased from 302 U/L to 162 U/L, providing a decreasing rate of 46%. Therefore, Composition A1 is beneficial to repair liver functions.

(2) Administration for 6 Months

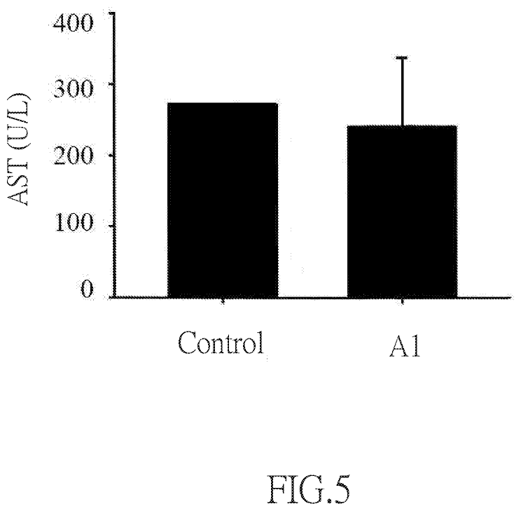

As shown in FIG. 5, after administration of Composition A1 for six months, regarding the liver function, AST value showed a decreasing trend. Therefore, Composition A1 is beneficial to repair liver functions.

The above embodiments are only preferred embodiments of the present invention, not intended to limit the present invention in any aspect. Although the preferred embodiments are disclosed above, they are not intended to limit the present invention in any aspect. Those skilled in the art, without departing from the scope of the technical solution of the present invention, can utilize the above disclosure with some minor modifications to the content as equivalent variations or modifications of equivalent embodiments. Any modification within the spirit and principle of the present invention, made, equivalent substitutions, improvements, etc., should be included within the scope of the present invention.

1. A method for treating liver dysfunction comprising administering to a subject in need thereof an effective amount of a composition comprising ferrous amino acid chelate and a pharmaceutically acceptable carrier.

2. The method according to claim 1, wherein the chelating ratio of ferrous to amino acid of the ferrous amino acid chelate in the composition comprising ferrous amino acid chelate is between 1:1 and 1:4.

3. The method according to claim 1, wherein the chelating ratio of ferrous to amino acid of the ferrous amino acid chelate in the composition comprising ferrous amino acid chelate is between 1:1.5 and 1:2.5.

4. The method according to claim 1, wherein the effective amount of the composition comprising ferrous amino acid chelate is between 0.5 mg/kg/day and 4 mg/kg/day.

5. The method according to claim 1, wherein the composition comprising ferrous amino acid chelate is prepared by mixing inorganic iron and amino acid and heating at 60° C. to 90° C. for 8 hours to 48 hours to obtain the composition comprising ferrous amino acid chelate, wherein the weight ratio of inorganic iron to amino acid is between 1:1.2 and 1:1.5.

6. The method according to claim 5, wherein the inorganic iron is ferrous sulfate, ferrous chloride or ferrous pyrophosphate, and the amino acid is glycine.

7. The method according to claim 5, wherein the composition comprising ferrous amino acid chelate further includes a reductant, wherein the reductant is ascorbic acid, citric acid, acetic acid, propionic acid, butyric acid, lactic acid, malic acid, sulfonic acid, succinic acid or any combination thereof.

8. The method according to claim 1, wherein the medicament is in an enteral or parenteral dosage form.

9. The method according to claim 8, wherein the enteral dosage form is an oral dosage form, wherein the oral dosage form is a solution, emulsion, suspension, powder, tablet, pill, lozenge, troche, chewing gum, or capsule.
